(19) United States
(12) Patent Application Publication
(10) Pub. No.: US 2026/0306705 A1
(43) Pub. Date: Oct. 8, 2026

(54) HYBRID THREE-DIMENSIONAL PRINTING AND ELECTROSPINNING PLATFORM FOR MULTI-LAYERED BIOMIMETIC SCAFFOLDS

(71) Applicants: Wright State University, Dayton, OH (US); Marshall University Research Corporation, Huntington, WV (US)

(72) Inventors: Nasim NOSOUDI, Huntington, WV (US); Gary COFFMAN, Lavalette, WV (US); Adam CUNNINGHAM, Circleville, OH (US)

(21) Appl. No.: 19/638,705

(22) Filed: Apr. 3, 2026

Related U.S. Application Data
(60) Provisional application No. 63/783,284, filed on Apr. 4, 2025.

(51) Int. Cl. A61L 27/18; A61L 27/56; B29C 64/106; B29C 64/209; B29C 64/245; B29C 64/314; B29C 64/393; B33Y 10/00; B33Y 30/00; B33Y 70/00; B33Y 80/00; C08L 67/04

(52) U.S. Cl. CPC A61L 27/18; A61L 27/56; B29C 64/106; B29C 64/209; B29C 64/245; B29C 64/314; B29C 64/393; B33Y 10/00; B33Y 30/00; B33Y 70/00; B33Y 80/00; C08L 67/04; A61L 2400/12

(57) ABSTRACT

Example embodiments disclose a hybrid three-dimensional (3D) printing and electrospinning system that includes a 3D printer configured to print biomaterial, the 3D printer includes a printer head assembly for extruding a liquid solution that forms the biomaterial via a nozzle, an electrospinning unit for generating electrospin fibers, wherein the printed biomaterials and electrospin fibers are extruded from the same nozzle, and a collection plate for assembling the printed biomaterial and electrospun fibers.

### CROSS-REFERENCE TO RELATED APPLICATION

The present application claims benefit to U.S. Provisional Application 63/783,284, filed Apr. 4, 2025, of which is incorporated by reference herein in its entirety.

### STATEMENT REGARDING FEDERALLY SPONSORED RESEARCH

This invention was made with government support under grant number 2337961 awarded by the National Science Foundation (NSF). The government has certain rights in the invention.

### TECHNICAL FIELD

The present disclosure relates generally to biomimetic scaffolds. More specifically, the present disclosure relates to a hybrid three-dimensional (3D) printing and electrospinning platform for multi-layered biomimetic scaffolds.

### BACKGROUND

Tissue engineering aims to create biomimetic scaffolds that replicate structural and functional properties of native tissues to support cell growth and regeneration. A critical challenge in this field is fabricating complex, hierarchical scaffolds that integrate micro-and nanoscale features to mimic an extracellular matrix (ECM). Traditional methods, such as solvent casting, freeze-drying, and gas foaming, often fall short in achieving the combination of macroscale structure, microscale porosity, and nanoscale topography necessary to mimic native tissues.

Electrospinning has emerged as a widely used technique for producing nanofibrous scaffolds with ECM-like features, including high surface-area-to-volume ratios, tunable porosity, and favorable cell-material interactions. Electrospun fibers provide a biomimetic microenvironment that promotes cell adhesion, proliferation, and differentiation. However, despite its advantages, conventional electrospinning alone struggles to produce scaffolds with precise three-dimensional geometries and controlled macro-architecture, which are essential for functional tissue regeneration. To address this limitation, recent efforts have focused on integrating 3D printing and electrospinning to combine the macroscale precision of additive manufacturing with the nanoscale fidelity of electrospinning. For instance, one conventional approach developed a hybrid platform that combined fused deposition modeling (FDM) with electrospinning to fabricate hierarchical scaffolds, demonstrating enhanced mechanical properties and biomimetic features. The process was sequential but not simultaneous meaning the electrospinning and extrusion functions were performed through separate nozzles and controlled independently. Similarly, another conventional approach fabricated hybrid polycaprolactone (PCL) scaffolds using a combination of 3D printing and electrospinning, achieving multi-scale structures that promoted osteogenic differentiation in vitro. However, in this approach, the 3D printing and electrospinning were not performed through the same nozzle. Instead, this approach used a sequential, layer-by-layer technique, alternating between two different systems. Despite significant progress in hybrid fabrication approaches, the vast majority of existing systems employ separate nozzles or even entirely separate machines for 3D printing and electrospinning, resulting in process discontinuity and potential challenges in fiber integration, interlayer adhesion, and scaffold uniformity.

Accordingly, there remains a need to develop a hybrid three-dimensional (3D) printing and electrospinning platform for multi-layered biomimetic scaffolds to resolve the issues of existing systems.

### SUMMARY

In an exemplary embodiment, a hybrid three-dimensional (3D) printing and electrospinning system is disclosed, which includes a 3D printer configured to print biomaterial, the 3D printer includes a printer head assembly for extruding a liquid solution that forms the biomaterial via a nozzle, an electrospinning unit for generating electrospin fibers, wherein the electrospin fibers are extruded from the same nozzle, and a collection plate for assembling the printed biomaterial and electrospun fibers.

In a further exemplary embodiment, a method for fabricating a fibrous construct is disclosed, which includes printing a first biomaterial layer including a micro-pattern for producing at least one microstructure onto a first layer of fibrous material, electrospinning nanofibers directly on the first layer of fibrous material, and generating the fibrous construct patterned with the printed and electrospun first layer of fibrous material, forming a biomimetic scaffold with hierarchical structure, wherein the printing and the electrospinning are performed on a same platform with a single nozzle.

In a further exemplary embodiment, a fibrous biomimetic scaffold construct is disclosed, comprising a hierarchical structure defined by a plurality of biomaterial layers comprising at least a first layer and a second layer, at least one of the plurality of layers comprising a microstructure of fibrous material having a grid-like architecture, and at least one of the plurality of layers comprising electrospun nanofibers, the layers arranged from bottom to top in any order, the second layer formed directly on the first layer.

In some embodiments, a fibrous biomimetic scaffold construct is disclosed, comprising a hierarchical structure defined by a plurality of biomaterial layers comprising at least a first layer and a second layer, the layers arranged from bottom to top, the second layer formed directly on the first layer, the first layer comprising a microstructure of fibrous material having a grid-like architecture, and the second layer comprising electrospun nanofibers deposited within and around the grid-like microstructure of fibrous material to provide a highly porous and interconnected fibrous network through the biomimetic scaffold construct.

In some embodiments, a fibrous biomimetic scaffold construct is disclosed, comprising a hierarchical structure defined by a plurality of biomaterial layers comprising three or more layers, the layers arranged from bottom to top, each consecutive layer formed directly on the adjacent lower layer, at least one of the layers comprising a microstructure of fibrous material having a grid-like architecture, and at least one of the layers comprising electrospun nanofibers, wherein at least one layer comprising electrospun nanofibers is deposited on the at least one layer comprising a microstructure of fibrous material having a grid-like architecture, wherein the electrospun nanofibers are deposited within and around the grid-like microstructure of fibrous material to provide a highly porous and interconnected fibrous network through the biomimetic scaffold construct.

In some embodiments the disclosure provides a biomimetic scaffold comprising at least three or more layers, the layers comprising one or more layers comprising electrospun nanofibers, and one or more layers comprising a grid-like microstructure of fibrous material, the layers arranged in an alternating fashion, wherein at least one of the layers comprising the electrospun nanofibers is deposited on top a layer comprising the grid-like microstructure of fibrous material.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the present invention will be obtained by reference to the following detailed description that sets forth illustrative embodiments, in which the principles of the invention are used, and the accompanying drawings of which:

FIG. 1 is a schematic diagram of an example hybrid three-dimensional (3D) printing and electrospinning system made in accordance with the present disclosure;

FIG. 2 is a perspective and magnified views of an example biomimetic scaffold which may be fabricated utilizing the hybrid three-dimensional (3D) printing and electrospinning system of FIG. 1, according to an example embodiment;

FIG. 3A is an image of a scaffold at a first time laid by the hybrid three-dimensional (3D) printing and electrospinning system of FIG. 1, according to an example embodiment;

FIG. 3B is a microscopic image of the scaffold of FIG. 2A at a second time laid by the hybrid three-dimensional (3D) printing and electrospinning system of FIG. 1, according to an example embodiment;

FIG. 3C is a microscopic image of the scaffold of FIG. 2A at a third time laid by the hybrid three-dimensional (3D) printing and electrospinning system of FIG. 1A, according to an example embodiment;

FIG. 4A is a low-magnification scanning electronic microscope (SEM) image showing a distinct interface between two components of a hybrid scaffold utilizing the hybrid three-dimensional (3D) printing and electrospinning system of FIG. 1, according to an example embodiment; and

FIG. 4B is a high-magnification scanning electronic microscope (SEM) image of a distinct interface between two components of a hybrid scaffold utilizing the hybrid three-dimensional (3D) printing and electrospinning system of FIG. 1, according to an example embodiment.

It should be noted that these Figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION OF EXAMPLE EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will be controlled.

The present disclosure relates to a multifunctional system configured to integrate 3D printing and electrospinning within a single nozzle system. By enabling real-time, sequential layer-by-layer deposition without scaffold relocation, this unified system offers improved nanofiber integration, interfacial bonding, and overall scaffold fidelity. As such, the present systems and methods achieve a single-nozzle hybrid fabrication, providing a versatile and efficient approach for creating multi-scale biomimetic scaffolds optimized for tissue engineering and regenerative medicine.

The present disclosure is based, in part, on the development of an innovative hybrid three-dimensional (3D) printing and electrospinning system which integrates additive manufacturing (AM) and electrospinning techniques to fabricate complex, multi-layered biomaterial scaffolds. The present systems and methods can sequentially print layers of biomaterial using a singular precision extrusion mechanism, followed by electrospinning of nanofibers directly onto the printed layer using the same deposition system. This alternating process of extrusion and electrospinning is repeated to create scaffolds with tunable structural and functional properties, mimicking the hierarchical architecture of native tissues. The resulting constructs demonstrate enhanced mechanical strength, controlled porosity, and a biomimetic microenvironment conducive to cell adhesion, proliferation, and differentiation. This hybrid fabrication approach opens new avenues for creating advanced scaffolds for tissue engineering and regenerative medicine applications, for example.

The present disclosure includes, in one aspect, a hybrid 3D printing and electrospinning system that integrates electrospinning with additive manufacturing using a singular material source. More specifically, the present disclosure includes a three-dimensional (3D) printer specially adapted to deposit alternating layers of biomaterial and nanofiber coatings when in use. In this regard, in some embodiments, the 3D printer includes a pneumatic syringe system which facilitates liquid biomaterial deposition, an air compressor with programmable valves to permit precise extrusion control of bioinks, a high-voltage electrospinning unit for generating nanofibers, a featureless conductive print bed (i.e., a collection plate) that ensures uniform electrospinning deposition and prevents structural misalignment, and software (programming instructions), which, when executed, controls layer-by-layer alternation between the bioprinting and electrospinning processes.

The present disclosure discloses an integration of an extrusion system with a high-voltage power supply to allow direct deposition of electrospun nanofibers onto a 3D printed layer from a same nozzle, creating scaffolds with hierarchical structures without damaging the 3D printer motor. This approach eliminates the need for separate fabrication systems and demonstrates the feasibility of producing multi-layered scaffolds with controlled macro-architecture from 3D printing and biomimetic fibrous microenvironments from electrospinning. This hybrid system establishes a versatile platform for engineering scaffolds that combine structural precision and cell-friendly topographies, suitable for applications in tissue engineering and regenerative medicine. Further, in some aspects, the present system allows precise deposition of 25% polycaprolactone (PCL) dissolved in tetrahydrofuran (THF), enabling seamless transitions between layer-by-layer 3D printing and electrospinning on the same scaffold.

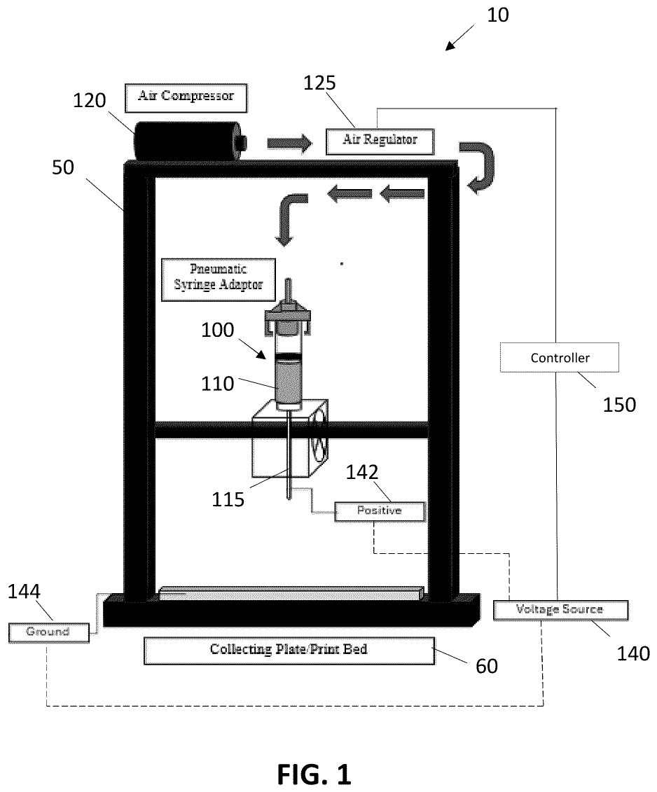

Referring now to the figures, FIG. 1 illustrates a bioprinter system 10 for producing 3D-printed biomaterials and electrospun fibers to construct a biomimetic scaffold, according to an example embodiment of the present disclosure. The system 10 includes a printer head assembly 100 for extruding a polymer liquid solution and a controller 150 for controlling an air compressor 120 via an air regulator 125 to control the printer head assembly 100 for 3D printing process and for controlling a positive terminal 142 of a high-voltage power supply 140 for an electrospinning process.

In some implementations, the liquid solution can be of a polycaprolactone (PCL) solution dissolved in pure tetrahydrofuran (THF) solution. In various aspects, the PCL solution can have concentrations of 10 grams (g), 15 g, 20 g, 25 g, or 30 g, each can be dissolved in 100 milliliters (mL) of THF solution. It is contemplated that an optimal concentration for the liquid solution can be PCL weighed 25 g and THF measured to 100 mL, establishing a 25% (w/v) solution. This liquid solution can be mixed using a magnetic stirrer and covered to keep the THF from evaporating while mixing. Once fully dissolved, the liquid solution can be stored in an airtight glass container where it could be accessed for use as needed. In other implementations, the polymer liquid can include other biomaterials, including natural polymers (e.g., collagen, gelatin) and composite systems.

As shown, the present system 10 includes a support frame 50 that houses or contains the printer head assembly 100 including a syringe system 110 for extruding the polymer liquid solution through a nozzle 115 onto a collection plate (deposition plate) 60. Optionally, it is contemplated that the printer head assembly 100 can comprise more than one syringe system 110. It is further contemplated that any required number of syringe system 110 can be used for a particular application. Unlike existing bioprinter systems, the present system 10 uses liquid solutions rather than solid filaments, in which case, a deposition plate of the existing system usually is coated with polyetherimide (PEI) to assist in adhesion of the filaments. However, one skilled in the art would appreciate that polyetherimide coating is incompatible with PCL-THF solution. Accordingly, to address this issue, the collection plate 60 of the present system 10 can be coated or formed as a zinc-plated stainless steel sheet. This ensures a smooth surface to produce precisely placed lines of the liquid solution, making retrieval easier and creating more accurate prints. In some implementations, the zinc-plated stainless-steel sheet of the collection plate 60 can include a small layer of a build platform stick glue to ensure constant adhesion during the 3D printing and electrospinning processes (even though the stand-alone zinc-plated stainless-steel sheet exhibited proper adhesion and uniformity).

In some implementations, the syringe system 110 can be a pneumatic subsystem, a hydraulic subsystem, a motorized linear subsystem, or a manually-powered linear subsystem for extruding the liquid solution through the nozzle 115. These subsystems can be operable by the air regulator 125 controlled by the controller 150. In some implementations, the syringe system 110 can be movable including a motor component (not shown) that is configured to generate movement(s) of the syringe system 110.

It is contemplated that the air compressor 120 and the air regulator 125 provide efficient power distribution and less electrical damage from a power supply as compared to a typical syringe pump associated with an existing system. It is further contemplated that the air compressor 120 and air regulator 125 can be mounted to a portion of the support frame 50 without occupying additional space. In some implementations, the air compressor 120 can be connected to the air regulator 125 via a properly sized thread adapter.

In one examplary setup, an optimal pressure of the air compressor 120 was 200 to 300 kilopascals (kPa), allowing for adequate extrusion of the liquid solution from the nozzle 115 and without a back pressure automatically shutting off the air compressor 120 once reaching its target pressure. In examplary aspects, the air regulator 125 was manually opened (via programmable valve(s)) sufficiently to allow air pressure to move through the syringe system 110 and extrude the liquid solution. In other examplary aspects, the air compressor 120 can reach about 60 to 70 kPa due to an amount of discharge created by the air regulator 125, ensuring that adequate pressure passes through the syringe system 110 and that there is accommodation for variations in pressure caused by environmental disturbance.

In some implementations, the nozzle 115 can be a needle, which is relatively a small diameter and designed with flexibility. In some aspects, the needle 115 can be a 20-gauge or 22-gauge needle. It is contemplated that the needle gauge can be varied depending on the desired deposition size, and thus is not limited to these specific examples. More generally, the nozzle may comprise a metallic needle of varying gauge suitable for controlled extrusion and electrospinning. It is further contemplated that the size of the nozzle 115 enabled the passing of liquid solution through the nozzle 115 without damaging the electronics. It is further contemplated, for proper electrospinning, metallic components such as springs, rods, and gears are not needed to ensure the electrospinning process was not affected as charge could jump between these components resulting in lack of sufficient voltage through the nozzle 115.

In other implementations, a filament detector system can be attached via wiring to the printer head assembly 100. This feature could either be turned off in the settings or a small piece of filament could be placed inside the system to indicate that the filament is present.

In various embodiments, the disclosure provides the means to prepare embodiments of a resultant biomimetic scaffold comprising a hierarchical structure defined by a plurality of biomaterial layers comprising at least a first layer and a second layer, at least one of the plurality of layers comprising a microstructure of material having a grid-like architecture, and at least one of the plurality of layers comprising electrospun nanofibers, the layers arranged from bottom to top in any order, the second layer formed directly on the first layer. According to some embodiments, a layer comprising electrospun nanofibers is deposited within and around a layer comprising the grid-like microstructure of fibrous material and provides a highly porous and interconnected fibrous network through the biomimetic scaffold construct.

In some embodiments, the disclosure provides the a fibrous biomimetic scaffold construct comprising a hierarchical structure defined by a plurality of biomaterial layers comprising at least a first layer and a second layer, at least one of the plurality of layers comprising a microstructure of fibrous material having a grid-like architecture, and at least one of the plurality of layers comprising electrospun nanofibers, the layers arranged from bottom to top in any order, the second layer formed directly on the first layer.

In some embodiments, the disclosure provides the a fibrous biomimetic scaffold construct comprising a hierarchical structure defined by a plurality of biomaterial layers comprising at least a first layer and a second layer, the layers arranged from bottom to top, the second layer formed directly on the first layer, the first layer comprising a microstructure of fibrous material having a grid-like architecture, and the second layer comprising electrospun nanofibers deposited within and around the grid-like microstructure of fibrous material to provide a highly porous and interconnected fibrous network through the biomimetic scaffold construct.

In some embodiments, the disclosure provides the a fibrous biomimetic scaffold construct comprising a hierarchical structure defined by a plurality of biomaterial layers comprising three or more layers, the layers arranged from bottom to top, each consecutive layer formed directly on the adjacent lower layer, at least one of the layers comprising a microstructure of fibrous material having a grid-like architecture, and at least one of the layers comprising electrospun nanofibers, wherein at least one layer comprising electrospun nanofibers is deposited on the at least one layer comprising a microstructure of fibrous material having a grid-like architecture, wherein the electrospun nanofibers are deposited within and around the grid-like microstructure of fibrous material to provide a highly porous and interconnected fibrous network through the biomimetic scaffold construct.

In some embodiments the disclosure provides a biomimetic scaffold comprising at least three or more layers, the layers comprising one or more layers comprising electrospun nanofibers, and one or more layers comprising a grid-like microstructure of fibrous material, the layers arranged in an alternating fashion, wherein at least one of the layers comprising the electrospun nanofibers is deposited on top a layer comprising the grid-like microstructure of fibrous material.

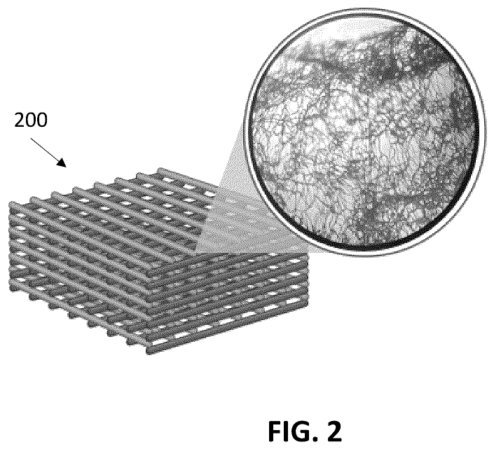

FIG. 2 illustrates a fabricated multilayered, biomimetic scaffolds with hierarchical structure 200, according to an example embodiment of the present disclosure. To fabricate the biomimetic scaffold 200, liquid biomaterial (e.g., by non-limiting example, polycaprolactone (PCL) in tetrahydrofuran (THF)) can be extruded via the syringe system 110 (and nozzle 115) onto the collection plate 60 to form a bioprinted macrostructure. During this process, the air compressor 120 and/or the programmable valves of the air regulator 125 may be adjusted to control the deposition speed and layer thickness of the biomaterial. Once the 3D macrostructure is formed, the high-voltage power supply 140 can be activated to cause the system to electrospin nanofibers via needle 115 directly onto the bioprinted macrostructure. For example, in one implementation, after a desired number of layers of the 3D printed structure is laid onto the collection plate 60, the printer head assembly 100 and the air compressor 120 are powered off and the power source 140 turned on to control the positive wire 142 to the syringe needle 115 and the ground wire 144 to the collection plate 60 for the electrospinning process. In some implementations, the printer head assembly 100 with the syringe system 110 can be moved to allow for adequate distance for the liquid solution to change phases from liquid to solid fibers. In one implementation, the printer head assembly 100 can be moved to a height of approximately 10 to 15 centimeters from its initial position. Once the printer head assembly 100 is adjusted to the appropriate height, the air compressor 120 is turned on. Once the solution starts forming a droplet at the tip of the needle, the power supply is turned on between 12 to 15 kilovolts (kV), for example. It is appreciated that power supply described herein is merely exemplary and non-limiting and other power supply voltage can be employed depending on the desired application. After the electrospinning process is completed, the printer head assembly 100 is adjusted back (move) to its original height and the 3D printing process resumes or repeated. As such, this enables both 3D printing and electrospinning within a single setup, i.e., via same needle or nozzle 115.

It is contemplated that the alignment and deposition of the fibers during the electrospinning process may be controlled by adjusting the electric field and/or air pressure settings. The foregoing bioprinting-electrospinning process is repeated in alternating fashion until a scaffold possessing a desired microscale and nanoscale architecture is produced, as represented by FIG. 2. It is further contemplated that the 3D printing process mostly determines the height of the final object since the 3D printed layer has the substantial thickness or height. For example, the 3D printed layer can have a height of approximately 0.2 mm, while the electrospun layer can have a height of approximately less than 100 μm. It is appreciated that thickness and/or height layer described herein is merely exemplary and non-limiting and other thickness and/or height layer can be employed depending on the desired application.

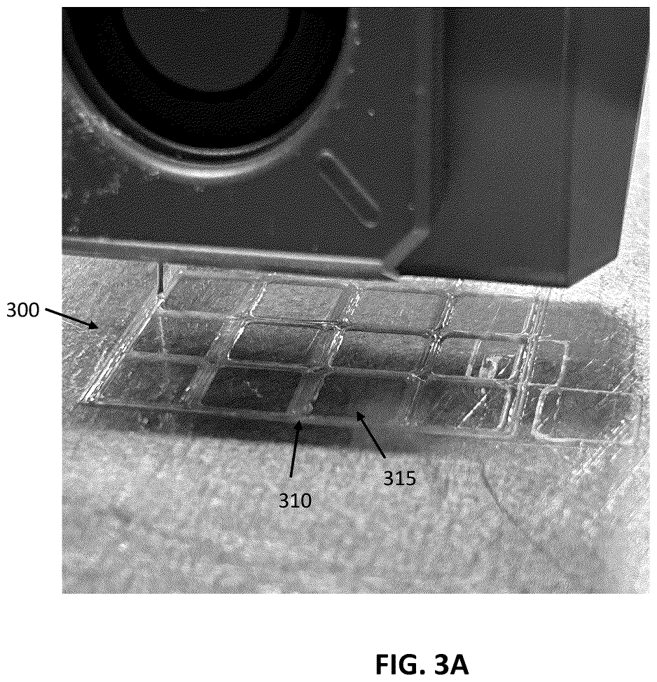

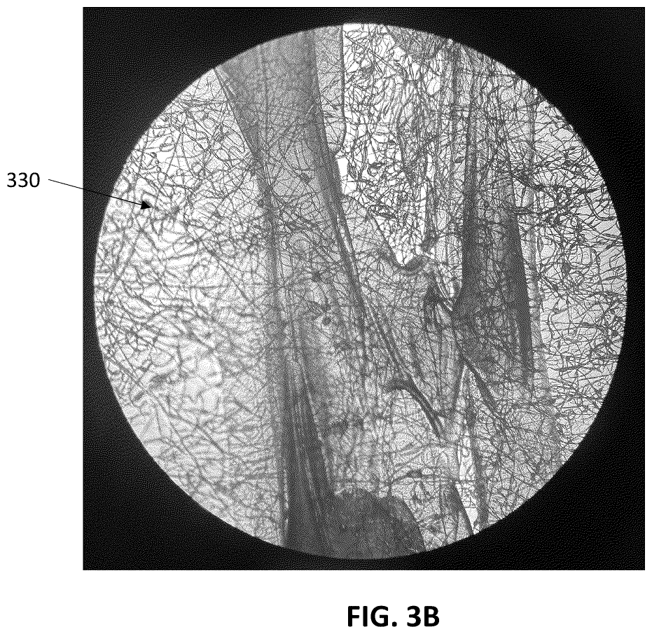

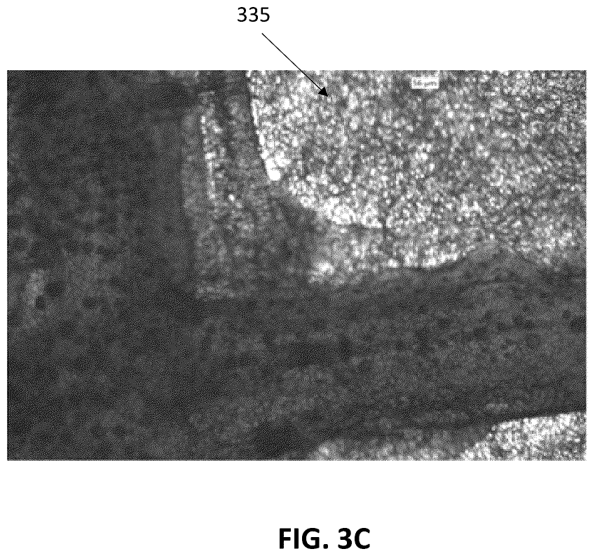

FIG. 3A is an image of a scaffold 300 at a first time, where the scaffold 300 includes two layers of polycaprolactone (PCL) in tetrahydrofuran (THF) (PCL-THF) laid by the hybrid three-dimensional (3D) printing and electrospinning system 10, according to an example embodiment. The scaffold 300 is fabricated using the hybrid 3D printing and electrospinning system 10 to integrate structural precision with nanoscale fibrous features. As shown, the hybrid 3D-printed framework provides a well-defined grid-like architecture 310, while electrospinning deposited a secondary fibrous layer 315 within and around the 3D printed structures 310, resulting in a multi-scale scaffold 300 designed to enhance mechanical stability and biological performance.

FIG. 3B is a microscopic image (2× magnification) of the scaffold 300 of FIG. 3A at a second time, where the scaffold 300 includes a layer of electrospun nanofibers 330 deposited onto two layers of PCL-THF by the hybrid three-dimensional (3D) printing and electrospinning system 10, according to an example embodiment.

FIG. 3C is a microscopic image (2× magnification) of the scaffold 300 of FIG. 3A at a third time, where the scaffold 300 includes two additional of (PCL-THF) and an additional of electrospun nanofibers 335 deposited onto the scaffold 300 shown in FIG. 3B by the hybrid three-dimensional (3D) printing and electrospinning system 10, according to an example embodiment.

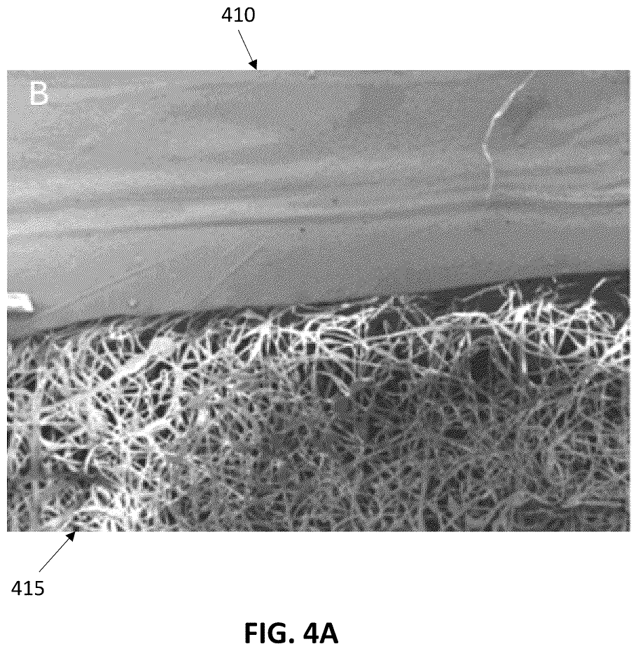

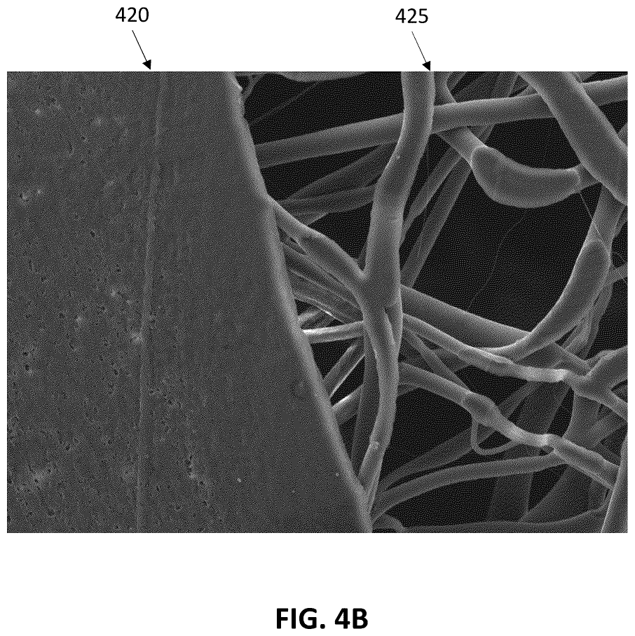

FIG. 4A is a low-magnification (50× magnification) scanning electronic microscope (SEM) image showing a distinct interface between two components 410, 415 of a hybrid scaffold utilizing the hybrid three-dimensional (3D) printing and electrospinning system of FIG. 1A, according to an example embodiment. As shown, this SEM image reveals a highly porous and interconnected fibrous network 415 filling the 3D-printed framework 410. Accordingly, this hierarchical structure is expected to facilitate nutrient transport, oxygen diffusion, and cell infiltration across multiple scales.

FIG. 4B is a high-magnification (500× magnification) scanning electronic microscope (SEM) image showing a distinct interface between two components of a hybrid scaffold utilizing the hybrid three-dimensional (3D) printing and electrospinning system of FIG. 1A, according to an example embodiment. As shown, this SEM image confirms the formation of smooth, bead-free electrospun fibers tightly integrated with the printed framework. More specifically, on the left side of the image, a dense, relatively smooth region 420 represents the 3D-printed biomaterial, showcasing the controlled deposition achievable through additive manufacturing. This region 420 provides structural support and mechanical integrity to the overall scaffold. Transitioning from this dense region 420, a network of randomly oriented fibers 425 is visible, characteristic of an electrospun porous scaffold. These fibers 425, with diameters on the micrometer scale (as indicated by the 10 μm scale bar), create a highly interconnected porous structure. This porosity is crucial for cellular infiltration, nutrient transport, and tissue regeneration within the scaffold. The SEM image of FIG. 4B clearly illustrates the seamless integration of these two distinct materials, highlighting the potential of the hybrid fabrication approach to create scaffolds with tailored structural and functional properties.

In some implementations, quantitative analysis of randomly selected fibers demonstrated an average fiber diameter of 5.86±0.93 μm, indicating moderate heterogeneity in fiber size, which supports diverse cell attachment and migration patterns. Further, such diversity in fiber dimensions can create microenvironments that promote cell adhesion, proliferation, and migration, thereby enhancing the biological performance of the scaffolds. It is appreciated that the fiber diameter described herein is merely exemplary and non-limiting and other fiber diameter can be employed depending on the desired application.

As shown by FIGS. 4A and 4B, these images show a distinct interface between the dense printed biomaterial region and the porous electrospun fiber region, supporting fidelity/registration of alternating layers. Accordingly, some attributes such as enhanced mechanical strength and controlled porosity and/or embedded micro/nanofiber scale approximately 10-30 μm (noted as relevant for infiltration/transport) are provided. Overall, the successful integration of 3D printing and electrospinning produced scaffolds with precise macroscopic geometry and biomimetic fibrous microenvironments, making them promising candidates for tissue engineering and regenerative medicine applications.

It is contemplated that example embodiments described by the present disclosure disclose a hierarchical architecture of biomimetic scaffolds for a variety of tissue engineering applications. For instance, the integration of macro-and nanoscale features provides both mechanical support and cell-friendly microenvironments, making them particularly suitable for: cartilage repair, where nanofibrous layers mimic collagen fibrils to support chondrocyte attachment; bone regeneration, as the robust printed framework provides mechanical stability while nanofibers enhance osteogenic differentiation; and/or soft tissue engineering, where interconnected porous networks can facilitate vascularization and nutrient transport. By optimizing scaffold structure at multiple scales, this platform bridges the gap between biomaterial design and functional tissue regeneration.

In some implementations, if necessary, post-processing and/or characterization steps may be carried out. In this regard, the scaffold fabricated by the present system may be ultraviolet (UV) cross-linked or chemically stabilized. In some instances, the scaffold may be analyzed via scanning electron microscopy (SEM) to confirm fiber morphology and mechanical testing for durability.

### Exemplary Systems and Methods

In some embodiments, a 3D printer such as a Neptune 3 Max 3D Printer manufactured by ELEGOO of Shenzhen, Guangdong Province, China, was used as a base system for the fabrication of a dual 3D printed and electrospun scaffolds. In some aspects, the hybrid scaffolds were composed of successive layers of 3D printed biomaterial and electrospun solution, forming a multi-layered structure. To form the printed biomaterial, the 3D printer extruded a polymer liquid solution of 25% solution of polycaprolactone (PCL) in tetrahydrofuran (THF) via nozzle 115 onto a collection plate 60, which includes a featureless surface (i.e., no grainy texture/surface) so as to ensure a smooth even deposition. In some aspects, the formation of the biomaterial process was conducted at approximately room temperature, and formation of the deposited structure was essentially instantaneous upon deposition. However, in other embodiments, the system may operate under controlled temperature conditions. For example, the nozzle and collection plate may be maintained at different temperatures, such as approximately 110° C. and 50° C., respectively, although other temperature settings may be used depending on the material system. The size and rate of the deposition can be controlled by a two-step programmable air compressor-manually controlled air regulator 125 connected to a pneumatic syringe adaptor for precise material extrusion. For example, in some implementations, the applied air pressure in one setup was approximately 6-10 psi, depending on the desired deposition speed. The deposited filament or line size depends on both the needle gauge and the 3D printing deposition speed, both of which are adjustable. For example, using a 22-gauge needle, the smallest deposited line achieved was approximately 0.5 mm after settling. The layer height is strongly dependent on the viscosity of the material; with our current solution, a layer height of approximately 0.2 mm was readily achieved. It is contemplated that more viscous materials may support greater layer height, while less viscous materials may spread more during deposition. In general, parameters including needle gauge, pressure, deposition speed, and resulting filament dimensions can be adjusted depending on the material system and desired scaffold characteristics.

After ensuring the 3D deposition features were functional, a high-voltage power supply was incorporated to facilitate the electrospinning process. In some aspects, the electrospinning solution can be extruded via the printed head assembly with 5-inch, 22-gauge needle at 0.15 mL/min at total fiber deposition time of 1 min. During this stage, the applied voltage was 12 to 15 kilovolts (kV), and the collection plate 60 with the printed biomaterial was maintained at 10 to 15 cm. It is contemplated that the driving forces of the electrospinning stage include the flow rate and the distance between the point where the high voltage is applied and the grounding of the collection plate 60. These two variables were kept constant throughout the manufacturing process. Then all scaffolds were kept at 40° C. for 10 min on the platform to ensure that no solvent remained. It is contemplated that the resulting scaffolds were homogeneous and repetitive; therefore, identical results were achieved in the characterization of the scaffolds manufactured in the different fabrication series.

In some aspects, the 3D printer was utilized to lay two layers of the PCL-THF solutions, creating a raised portion of material (FIG. 3A). After electrospinning onto the 3D printed scaffold, a portion of the sample was placed under a microscope set to 2x magnification (FIG. 3B). Next, an additional two layers of the solution was 3D printed onto the original sample followed by an additional layer of electrospinning and placed under microscope (2× magnification) (FIG. 3C).

Example embodiments disclose a hybrid 3D printing and electrospinning system that provides a fabrication of multi-layered scaffolds with alternating biomaterial layers and nanofiber coatings, mimicking the hierarchical structure of the extracellular matrix. This structure can be a significant development for therapeutic applications in tissue engineering and regenerative medicine. Accordingly, this enables the deposition of nanofibers directly onto the 3D printed layers, creating micro-to nanoscale fibers on the surfaces of the scaffolds. Combining these processes allows the system to alternately print biomaterial layers and electrospun nanofibers together.

Now to the discussion of executing a software to control the layer-by-layer alternation between the 3D bioprinting and electrospinning methods as described herein. As shown in FIG. 1, the controller 150, provided in system 10, is communicatively coupled to each of the high-voltage power supply 140 and/or programmable valves of the air regulator 125. The controller 150 can be operably connected to the one or more processors, such that operation of the high-voltage power supply 140 and/or the air regulator 125 can be regulated by the one or more processors to control the electrospinning and biomaterial depositions, respectively.

The controller 150 may be a computing system that includes a processor and a storage system. The controller 150 may include one or more processors (e.g., central processing units(s) (CPU(s)), graphics processing unit(s) (GPU(s)), and/or application-specific integrated circuit(s) (ASIC(s))), where the one or more processors are operable to execute instructions corresponding to the software stored in associated memory. The storage system includes software, including stored data, including data in database structure. Although the controller 150 is depicted in FIG. 1 as a single, unitary system encapsulating one processor and one storage system, it should be appreciated that more than one storage system and more than one processor, may comprise the controller. In some implementations, the controller 150 can be app or a cloud computing application and systems, including such as, on-premise infrastructure, edge computing, decentralized system, and/or localized hosting.

The processor(s) may be a microprocessor, a general-purpose central processing unit, an application-specific processor, a microcontroller, or any type of logic device. The processor(s) may also include circuitry for retrieving and executing software. For example, the processor(s) may have instructions to operate the high-voltage power supply 140 and/or programmable valves of the air regulator 125 by controlling durations and speeds of electrospinning and biomaterial depositions, respectively. It should be appreciated that the processor can alternatively be implemented with a single processing device, but may also be distributed across multiple processing devices or subsystems that cooperate in executing software instructions.

The storage system includes any storage media, or group of storage media, readable by the processor, and capable of storing software and data. In one implementation, the processor loads and executes software, including the operations of the high-voltage power supply 140 and/or the air regulator 125, which is a software application stored in the storage system. The processor accesses data stored in the database in order to carry out the methods and control instructions described herein. The storage system can include volatile and non-volatile, removable and non-removable media implemented in any method or technology for storage of information, such as computer-readable instructions, data structures, program modules, or other data. The storage system may store a set of processor instructions or algorithms, which when executed by the controller 150 enables automatic operation of the system 10. Examples of the non-volatile memory may include, but are not limited to, a flash memory, a Read Only Memory (ROM), a Programmable ROM (PROM), Erasable PROM (EPROM), and Electrically EPROM (EEPROM) memory. Examples of volatile memory may include, but are not limited Dynamic Random Access Memory (DRAM) and Static Random-Access memory (SRAM).

As to operation, the controller 150 provides control instructions to the programmable valves of the air regulator 125 and/or the high-voltage power supply 140, which controls the 3D biomaterial deposition and electrospinning, respectively. For example, the controller 150 operates the regulation of valves of the air regulator 125 at a particular speed to allow for the deposition of liquid materials via the pneumatic syringe adaptor and enable precise material extrusion. After ensuring the 3D biomaterial deposition features were functional, the controller 150 operates the high-voltage power supply 40 to control the positive terminal 141 and ground terminal 144 in the collecting plate 60 to permit the electrospinning process. This allows the deposition of nanofibers directly onto the 3D printed layers, creating micro-to nanoscale fibers on the surfaces of the scaffold.

It should be appreciated that specifications of the controller are not limited to the embodiments described herein, but may vary depending on the requirements of the present system.

While the terms used herein are believed to be well understood by those of ordinary skill in the art, certain definitions are set forth to facilitate explanation of the presently disclosed subject matter. Such specific terms are:
Biocompatible: A term describing something that can be substantially non-toxic in the in vivo environment of its intended use, and is not substantially rejected by the patient's physiological system. This can be gauged by the ability of a material to pass the biocompatibility tests set forth in International Standards Organization (ISO) Standard and/or the U.S. Pharmacopeia (USP) and/or the U.S. Food and Drug Administration (FDA). Typically, these tests measure a material's toxicity, infectivity, pyrogenicity, irritation potential, reactivity, hemolytic activity, carcinogenicity and/or immunogenicity. A biocompatible structure or material, when introduced into a majority of subjects, will not cause a significantly adverse reaction or response. Furthermore, biocompatibility can be affected by other contaminants such as prions, surfactants, oligonucleotides, and other agents or contaminants. The term “biocompatible material” refers to a material that does not cause toxic or injurious effects on a tissue or organ.
Layer: As used herein “layers”, “layering”, and “layered” are forms of the same term defining material and process for making a material where a first substance or substrate surface is at least partially covered or associated with a second substance. Both the first and second substance are not required to be different. Further, when a surface is “layered” as used herein, the layer may be effectuated by any chemical or mechanical bond or force, including linking agents. The “layers” need not be complete or cover the entire surface of the first substance to be “layered”. The “layering” may be complete as well (e.g., approximately covering the entire first substance). There can be multiple layers and multiple substances within each layer. The layering may vary in thickness or the layering thickness may be substantially uniform. Layering contemplated in accordance with the present disclosure include successive layers of 3D printed PCL filament and electrospun PCL solution.
Electrospinning or Electrospun: A process in which fibers are formed from a solution or melt by streaming an electrically charged solution or melt through an orifice or nozzle, typically in the nanometer to micrometer range.
Scaffold: A structural support facilitating cell infiltration and attachment in order to guide cell growth. As disclosed herein, a scaffold can be used to form a biomaterials containing cell or growth factors in tissue engineering.
Polymers: Common biodegradable polymers include polycaprolactone (PCL), poly(lactic-co-glycolic acid) (PLGA), and poly(vinyl alcohol) (PVA).

All patents, patent applications, published applications and publications, GenBank sequences, databases, websites and other published materials referred to throughout the entire disclosure herein, unless noted otherwise, are incorporated by reference in their entirety.
Actuator: Refers to any conventional device that is configured to effect axial movement of an object relative to a translation axis. For example, it is contemplated that an actuator can comprise a linear actuator, such as, for example and without limitation, a pneumatic actuator, a hydraulic actuator, a motorized linear actuator, or a manually-powered linear actuator. It is understood that an actuator can comprise a motor component that is configured to generate movement of the actuator.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accordance with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or evaluating the presently disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The aspects and embodiments of the invention can be used alone or in combinations with each other. “At least one,” as used herein, means one or more and thus includes individual components as well as mixtures/combinations.

Although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that, if an element is referred to as being “connected” or “coupled” to another element, it can be directly connected, or coupled, to the other element or intervening elements may be present. In contrast, if an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A hybrid three-dimensional (3D) printing and electrospinning system, comprising:
a 3D printer configured to print biomaterial, the 3D printer includes a printer head assembly for extruding a liquid solution that forms the biomaterial via a nozzle;
an electrospinning unit for generating electrospin fibers, wherein the electrospin fibers are extruded from the nozzle; and
a collection plate for assembling the printed biomaterial and electrospun fibers.

2. The system of claim 1, wherein the 3D printer is configured to print biomaterial and electrospin fibers in alternating fashion.

3. The system of claim 1, wherein the 3D printer includes a syringe-based extrusion system and an air compressor.

4. The system of claim 3, wherein the 3D printer further includes an air regulator that is in fluid communication with the air compressor and the syringe-based extrusion system and is configured to control a deposition of the liquid solution.

5. The system of claim 3, wherein the printed biomaterial and electrospun fibers are extruded from the syringe-based extrusion system via the same nozzle.

6. The system of claim 3, wherein the syringe-based extrusion system is a pneumatic syringe extrusion system.

7. The system of claim 1, wherein the electrospinning unit is electrically connected to a power supply to control the nozzle for electrospinning, wherein the power supply is selectively activated to generate an electric field to produce electrospun fibers.

8. The system of claim 7, wherein the power supply controls activation of a positive terminal connected to the nozzle and a ground terminal connected to the collection plate.

9. The system of claim 8, wherein the power supply is selectively activated based on pre-programmed instructions stored in memory associated with one or more processors of the 3D printer.

10. The system of claim 1, wherein the liquid solution is polycaprolactone dissolved in tetrahydrofuran.

11. The system of claim 1, wherein the electrospinning unit directly deposits electrospin fibers onto the printed biomaterial to form a biomimetic scaffold with hierarchical structure.

12. A method for fabricating a fibrous construct, comprising:
printing a first biomaterial layer including a micro-pattern for producing at least one microstructure onto a first layer of fibrous material;
electrospinning nanofibers directly on the first layer of fibrous material; and
generating the fibrous construct patterned with the printed and electrospun first layer of fibrous material, forming a biomimetic scaffold with hierarchical structure,
wherein the printing and the electrospinning are performed on a same platform with a single nozzle.

13. The method of claim 12, further comprising:
printing a second biomaterial layer including a micro-pattern for producing at least one microstructure onto a second layer of fibrous material;
electrospinning nanofibers directly on the second layer of fibrous material;
generating the fibrous construct patterned with the printed and electrospun second layer of fibrous material; and
laying the printed and electrospun second layer of fibrous material over the printed and electrospun first layer of fibrous material, forming a multi-layered biomimetic scaffold.

14. The method of claim 12, further comprising assembling the first layer of fibrous material and the second layer of fibrous material on a collection plate located below the nozzle.

15. The method of claim 12, wherein the printed first biomaterial layer is formed by depositing a liquid solution via the nozzle, the liquid solution is polycaprolactone dissolved in tetrahydrofuran.

16. The method of claim 15, wherein depositing the liquid solution is performed by a syringe-based extruder system controlled by an air compressor, wherein the air compressor is regulated by an air regulator to adjust control of deposition speed and rate of the liquid solution.

17. The method of claim 12, wherein electrospinning the nanofibers further comprises activating a power supply to control the nozzle for electrospinning.

18. The method of claim 17, wherein activating the power supply further comprises:
activating a positive terminal attached to the nozzle to supply nanofibers directly onto the printed first biomaterial layer; and
activating a ground terminal attached to a collection plate.

19. A biomimetic scaffold comprising a hierarchical structure defined by a plurality of biomaterial layers comprising at least a first layer and a second layer, the layers arranged from bottom to top in any order, the second layer formed directly on the first layer, the plurality of layers including a second layer comprising electrospun nanofibers deposited within and around a first layer comprising a grid-like microstructure of fibrous material to provide a highly porous and interconnected fibrous network through the biomimetic scaffold layers.

20. The biomimetic scaffold according to claim 19, comprising at least three or more layers, the layers comprising one or more layers comprising electrospun nanofibers, and one or more layers comprising a grid-like microstructure of fibrous material, the layers arranged in an alternating fashion, wherein at least one of the layers comprising the electrospun nanofibers is deposited on top a layer comprising the grid-like microstructure of fibrous material.
